(19) United States
(12) Patent Application Publication
(10) Pub. No.: US 2025/0127549 A1
(43) Pub. Date: Apr. 24, 2025

(54) SURGICAL IMPACTOR

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Brett Bell, Mendon, UT (US); Ryan Sheehan, Pittsburgh, PA (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/916,101

(22) Filed: Oct. 15, 2024

Related U.S. Application Data
(60) Provisional application No. 63/544,682, filed on Oct. 18, 2023.

(51) Int. Cl. A61B 17/92; A61F 2/46

(52) U.S. Cl. CPC A61B 17/921; A61B 2017/924; A61F 2002/4627

(57) ABSTRACT

An impactor and methods of operation of an impactor. The impactor includes an electromagnetic component that has a stationary electromagnetic housing and a moving magnet actuator, a striker, and one or more strike plates. The striker is coupled to an object. The stationary electromagnetic housing includes a coil that receives an electric current. The moving magnet actuator includes one or more magnets. The magnets generate a high potential magnetic field that interacts with the electric current applied to the coil disposed within the stationary electromagnetic housing and trigger translation movement of the moving magnet actuator. The electromagnetic field, generated as a result of application of the electric current to the coil, forces the moving magnet actuator to translate. Translation of the moving magnet actuator causes the moving magnet actuator to strike at least one of the strike plates to thereby translate the striker.

### CROSS-REFERENCE TO RELATED APPLICATIONS

This is a non-provisional of, and claims the benefit of the filing date of, pending U.S. Provisional Patent Application No. 63/544,682, filed Oct. 18, 2023, entitled “Surgical Impactor,” the entirety of which application is incorporated by reference herein.

### TECHNICAL FIELD

The present disclosure is generally directed to a surgical impactor, and, in particular, to a surgical impactor having an electromagnetic component for generating of an impact energy.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to insure a proper fit between the patient's femur and the implant. In addition, the orthopedic implant such as, for example, the acetabular cup, may need to be impacted into proper position. Moreover, during removal of the broach from the patient's intramedullary canal, the broach may become struck within the patient's intramedullary canal.

A surgical impactor may be used for insertion and/or removal of implants. Some existing impactors rely on motors to deliver energy for such insertion/removal. Other impactors use solenoid-based systems that produce electromagnetic energy that is applied to deliver the necessary force to objects coupled to the impactor (e.g., tools, implants, etc.), and hence, perform requisite surgical procedures. The solenoid-based impactor system suffer from parasitic eddy currents created during application of currents to the solenoid components, which, in turn, reduces an amount of force that may be delivered as well as the impactors' overall efficiency. As such, it would be beneficial to provide a surgical impactor tool that has such eddy currents reduced.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In some examples, the present disclosure relates to an impactor. The impactor may include an electromagnetic component that may include a stationary electromagnetic housing and a moving magnet actuator component, a striker component, and one or more strike plates. The striker component may be configured to be coupled to an object. The stationary electromagnetic housing may include a coil configured to receive an electric current. The moving magnet actuator may include one or more magnets (e.g., four and/or any other number). The magnets may be configured to generate a high potential magnetic field that may be configured to interact with the electric current applied to the coil disposed within the stationary electromagnetic housing and trigger translation movement of the moving magnet actuator component. The electromagnetic field, generated as a result of application of the electric current to the coil, may force the moving magnet actuator component to translate along the striker component. Translation of the moving magnet actuator component may cause the moving magnet actuator component to strike at least one of the strike plates to thereby translate the striker component.

In some examples, the present disclosure relates to a method for using a surgical impactor (e.g., to position an implant in a bone, remove the implant from the bone, and/or broach a femoral canal prior to placing the implant, etc.). The surgical impactor may include an electromagnetic component including a stationary electromagnetic housing and a moving magnet actuator component having one or more magnets, a striker component configured to be coupled to an object, and one or more strike plates. The method may include applying an electric current to a coil disposed within the stationary electromagnetic housing, wherein one or more magnets may be configured to generate a high potential magnetic field that interacts with the electric current applied to the coil, thereby translating the moving magnet actuator component. The method may further include triggering, as a result of the applying, translation of striker component. An electromagnetic field, generated as a result of application of the electric current to the coil, may be configured to force the moving magnet actuator component to translate. Translation of the moving magnet actuator component may be configured to cause the moving magnet actuator component to strike at least one of the strike plates to thereby translate the striker component. The method may further include performing positioning of the implant in the bone and/or removing of the implant from the bone.

Examples of the present disclosure provide numerous advantages. For example, the current subject matter's use of sintered metal composite materials in the stationary electromagnetic housing and portions of the moving magnet actuator component may allow reduction in eddy currents that may be generated as a result of application of current to the coil. Since eddy currents are parasitic, their reduction may, in turn, reduce the loss of power that is applied to the striker component by the moving magnet actuator component, thereby increasing the force that the striker component may apply to an object (e.g., a tool, an implant, etc.).

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain features of the subject matter disclosed herein and, together with the description, help explain some of the principles associated with the disclosed implementations. In the drawings,

FIG. 1 illustrates an exemplary impactor that may be configured to incorporate a system for reducing electromagnetic eddy currents during operation of the impactor, in accordance with one or more features of the present disclosure;

FIGS. 2a-e illustrate further details of the impactor shown in FIG. 1, in accordance with one or more features of the present disclosure;

FIG. 3 illustrates another cross-sectional view of a portion of the impactor shown in FIG. 1, in accordance with one or more features of the present disclosure;

FIGS. 4a-c illustrate another exemplary impactor, in accordance with one or more features of the present disclosure;

FIG. 5 is a block diagram of an exemplary orthopedic surgical instrument and/or impactor in accordance with one or more features of the present disclosure;

FIG. 6 illustrates an exemplary computing apparatus in accordance with one or more features of the present disclosure;

FIG. 7 illustrates an example of a storage medium to store impactor logic in accordance with one or more features of the present disclosure; and

FIG. 8 illustrates an example computing platform in accordance with one or more features of the present disclosure.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide an orthopedic surgical instrument or impactor, and in particular, a surgical impactor tool that may be characterized by a reduced amount of electromagnetic eddy currents.

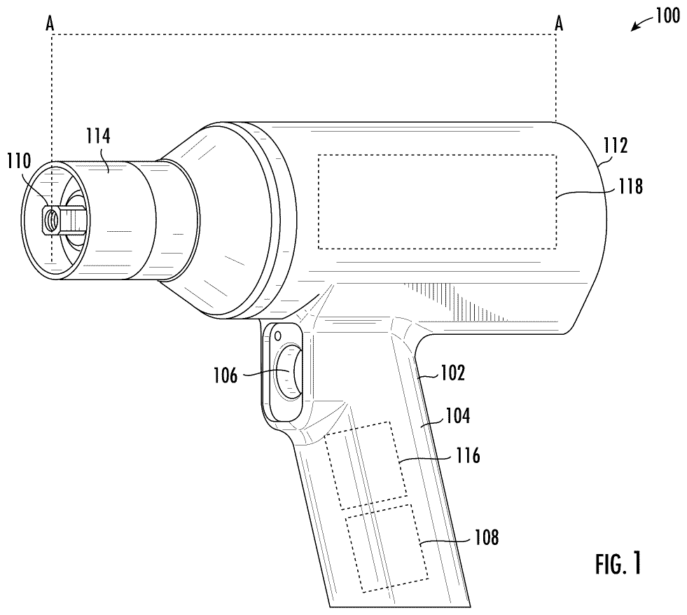

It should be appreciated that while, for example, reduction of electromagnetic eddy currents in accordance with the present disclosure may be described herein in connection with an orthopedic surgical impactor (e.g., as shown in FIG. 1) that may be used to, for instance, drive a broach into a patient's bone to, for example, prepare an intramedullary canal of the patient's bone, the present disclosure is not so limited and such reduction of electromagnetic eddy currents (as shown and described hereinbelow) may be used in connection with any tool, device, instrument, etc. now known or hereafter developed such as, for example, a tap used to insert a bone screw, construction hand tools or power tools, etc. As such, the present disclosure should not be limited to any particular tool unless explicitly claimed.

The following description of an orthopedic impactor (e.g., as shown in and discussed in connection with FIG. 1) is provided here for exemplary, illustrative purposes only and is not intended to limit the current subject matter and/or any of its elements, applications and/or advantages.

With reference to FIG. 1, an example of an orthopedic surgical instrument, impactor, or impactor mechanism (terms used interchangeably herein without the intent to limit or distinguish) is disclosed. In use, the orthopedic impactor is arranged and configured to transmit a forward energy or motion to, for example, drive a surgical tool (e.g., a broach) or implant into a patient's bone, and a reverse energy or motion to, for example, remove a struck or lodged surgical tool (e.g., a broach) or implant from a patient's bone.

The orthopedic impactor may be arranged and/or configured to position, insert and/or implant an orthopedic implant such as, for example, but not limited to, an acetabular cup, an intramedullary nail, a femoral hip implant, etc. into a bone matter (e.g., a bone of a patient). Alternatively, or in addition to, the orthopedic impactor may be coupled to a surgical tool such as, for example, but not limited to, a broach, to prepare a bone to receive an orthopedic implant.

The orthopedic impactor may cause application of a forward energy and/or motion to drive an orthopedic implant and/or surgical tool into a patient's bone. In addition, the orthopedic impactor may be arranged and/or configured to cause application of a reverse energy and/or motion to, for example, but not limited to, remove a stuck and/or lodged surgical tool and/or implant from a patient's bone. The orthopedic impactor may be configured to allow selection between the forward and/or reverse application of energy and/or motion by pushing forward and/or pulling back on the orthopedic impactor. During use, a user (e.g., a medical professional, a doctor, a surgeon, etc.) may push forward on an orthopedic impactor thereby causing a hammer of the impactor to strike a first and/or a forward impaction surface causing the orthopedic impactor to drive an orthopedic implant and/or surgical tool. Alternatively, or in addition, the user may pull back on the orthopedic impactor thereby causing the impactor's hammer to strike a second and/or a reverse impaction surface causing the orthopedic impactor to produce a reverse impaction to, for example, remove an orthopedic implant or surgical tool.

It should be appreciated that while, for example, the orthopedic impactor may be described herein in connection with driving a broach into a patient's bone to, for example, prepare an intramedullary canal of the patient's bone, the current subject matter is not so limited, and the orthopedic impactor may be used in connection with any surgical tool and/or implant now known and/or hereafter developed. As such, the current subject matter should not be limited to any particular surgical tool, device, instrument, implant, and/or procedure unless explicitly claimed. The orthopedic impactor may be arranged and/or configured to apply a force, while minimizing the risk of injury to the patient and/or to the user's hands during use. Moreover, the orthopedic impactor may be configured to assist its user to deliver a force towards and/or away from a surgical area in, for example, but not limited to, a joint replacement procedure.

In some implementations, the current subject matter relates to an impactor that may incorporate electromagnetic devices and/or systems for driving movement of its striker component and that may provide an enhanced application of power through reduction of parasitic electromagnetic eddy currents associated with such electromagnetic devices/systems. When electromagnetic devices experience changing magnetic fields, so-called eddy currents are generated (primarily) in ferrous materials. Since flux conducting pathways are ferrous, eddy currents represent a parasitic loss to the system in general. Specific to a solenoid operated surgical impactors, eddy currents are particularly relevant when current is sent to the solenoid which accelerates impactor's armature, which ultimately delivers the impact energy to the device attached to the striker component. Naturally, the more energy that can be imparted to the armature, the more energy is available for impaction.

Eddy currents are proportional to the change in magnetic flux, and thus, the initial in-rush of current creates a large flux differential, which, in turn, creates eddy currents. This also causes creation of a counter magnetomotive force, which slows the current in-rush, thereby delaying full development of the desired magnetic field. Additionally, any time, the current passes through a material, it generates heat, which may be destructive to the impactor. Thus, if eddy currents can be reduced, the heat generated by the device can also be reduced.

Conventionally, eddy current reduction is addressed in various ways. For example, one way to reduce eddy currents may involve reduction of electrical conductivity of ferrous materials used. This has a limited impact since altering electrical conductivity of ferrous materials also affects their magnetic properties. Another way is to introduce geometrical constraints which limit cross-sectional areas that the magnetic flux interacts with to reduce the magnetic gradient and, hence, the eddy current. It is used in many electromagnetic systems and devices that are subject to changing magnetic fields. Change of geometrical constraints typically involves forming thin laminations of a ferrous alloy, also called electrical steel, which has a high silicone content. Laminations are coated with a thin electrical insulator. This construction is very effective in reducing eddy currents to insignificant levels.

When solenoids are used, a flux path is axial in the armature and an outer sleeve. Since the induced current is always perpendicular to the changing flux, eddy currents are induced in a circumferentially oriented direction. The application of circumferentially oriented laminations becomes less effective since each lamination would need to be wedge shaped in its cross-section. Otherwise, large gaps may ensue due to the difference in circumference between inner and outer radii. The same issue exists for the armature. Alternatively, discrete V-shaped axial slots rather than laminations may be used, however, they are not nearly as effective due to the limited number of slots that can be practically made in the solenoid-based devices.

The current subject matter addresses the above problems with conventional methods through arrangement of various structural elements, which may include magnetic and non-magnetic components, as well as use of sintered composites materials for one or more of such components. Sintered composite materials use ferrous particles coated in a ceramic insulator. This allows these materials to retain magnetic properties while drastically reducing eddy currents. In particular, the sintered composite materials (e.g., sintered metal composite materials) may be used to form flux carrying elements in an electromagnet-based surgical device, such as, an impactor. As a result, the electromagnet-based impactor may be capable of faster current switching, thereby imparting more energy as well as generating less heat that is typically associated with eddy currents. Further, the sintered composite materials may provide an ability to create complex geometry for the components involved with relatively low cost. In the case of the solenoid, while the geometry may be relatively simple, use of sintered composite materials may enable advantageous use of their unique material properties in reducing eddy currents.

Thus, in some implementations, the current subject matter may relate to an impactor that may include an electromagnetic component that may include a stationary electromagnetic housing and a moving magnet actuator component, a striker component, and one or more strike plates, as well as various other components, as will be discussed herein. The striker component may be configured to be coupled to the strike plates. At a distal end, the striker component may also be coupled to an object. The object may include at least one of: a tool, an implant, and/or any other object, and/or any combination thereof.

In some example, non-limiting implementations, the moving magnet actuator may include one or more magnets (e.g., four and/or any other number). Each pair of magnets may have an opposite polarity to the other pair of magnets. The magnets may be configured to generate a high potential magnetic field in a pole component that may be configured to separate the pairs of magnets. The high potential magnetic field may be configured to interact with the electric current applied to a coil disposed within the stationary electromagnetic housing to trigger translation movement of the moving magnet actuator component. The electromagnetic field, generated as a result of application of the electric current to the coil, may force the moving magnet actuator component to translate along the striker component. Translation of the moving magnet actuator component may cause the moving magnet actuator component to strike at least one of the strike plates to thereby translate the striker component.

The moving magnet actuator component may include one or more pole components that may enclose the magnets. The pole components may be composed of one or more sintered composite materials, which may be sintered metal composite materials. The stationary electromagnetic housing (which may house the coil) may likewise be composed of one or more sintered composite materials. As stated above, use of the sintered composite materials may reduce eddy currents that may result from the axial electromagnetic flux.

In some implementations, the directions of translation movement of the moving magnet actuator component and the striker component may be interdependent, which in turn, may be dependent on a direction of the current applied to the coil within the stationary electromagnetic housing. Further, the direction of translation movement of the moving magnet actuator component may be directly and/or indirectly dependent on the direction of the current. Further, a change in the direction of the current applied to the moving magnet actuator component may change the direction of translation movement of the moving magnet actuator component, and hence, the striker component.

FIG. 1 illustrates an exemplary impactor 100 that may be configured to incorporate a system for reducing electromagnetic eddy currents during operation of the impactor (as shown and described below), according to some implementations of the current subject matter. The impactor 100 may be configured to include a housing 102, a handle 104, an actuator or a trigger assembly 106, a power source 108, a distal connector assembly 110, a strike mechanism housing 112, a distal connector housing 114, electronics 116, and a strike mechanism 118 (positioned within the strike mechanism housing 112). In some example, non-limiting implementations, the impactor 100 may also include a user interface, an electronic display and/or an input keypad and/or key panel (e.g., touch-based keypad/key panel) that may be used to display and/or allow the user to enter various operational parameters of the impactor 100.

The housing 102 may be configured to house and/or enclose one or more components of the impactor 100. The housing 102 may be manufactured from any suitable material now known or hereafter developed such as, for example, but not limited to, plastic, metal, composite material, fiberglass, and/or any combination thereof.

The housing 102 may include the handle portion 104 with an optional handgrip for comfortable and secure holding of the impactor 100 for use during a procedure (e.g., positioning of an implant into a bone). Alternatively, or in addition to, the housing 102 may incorporate a suitable mounting interface for integrating the impactor 100 into a robotic assembly during use. In some example implementations, the housing 102 may be a unitary structure and/or may include multiple components that may be assembled together.

The housing 102 may also include a reception port for receiving the power source or the battery (hereinafter, “battery”) 108. The battery 108 may be a rechargeable battery and may be removed from the housing 102 after use, such as, for example, for recharging. As can be understood, the battery 108 may recharged while coupled to the housing 102. Use of the battery 108 may provide for portability and versatility of the impactor 100 during use, i.e., the user of the impactor 100 does not have to be concerned with power wires (and/or pneumatic tubes) extending from the impactor 100. Alternatively, or in addition, the housing 102 may include one or more power ports (not shown in FIG. 1) that may be used to couple one or more power wires (e.g., to provide power and/or power in addition to the battery power 108) and/or one or more pneumatic tubes (e.g., to provide additional air pressure to the impactor 100 during use). As can be understood, more than one battery 108 may be included in the housing 102 and/or used during procedures. Any type of battery may be used, such as, for example, but not limited to, alkaline, nickel metal hydride (NiMH), lithium ion, and/or any combination thereof. The battery 108 may be incorporated and/or inserted into the handle 104 and/or coupled to the handle 104 (FIG. 1 illustrates the battery 108 being incorporated and/or inserted into the handle 104). As can be understood, other ways of coupling and/or connecting the battery 108 to electrical circuits of the impactor 100 are possible.

In some example, non-limiting implementations, the battery 108 may be at least one of the following: a capacitor, a supercapacitor, an interface for receiving mains power, an external power source, a chemical power source, and/or any combinations thereof. Further, combining a battery and/or a capacitor/supercapacitor may enable provision of a large power source that may, via charging of a capacitor, provide a large instantaneous and/or short-term current. Alternatively, or in addition, the impactor 100 may be powered using any type of power source, such as, for example, but not limited to, alternating current (AC) power source, direct current (DC) power source, and/or any combination thereof, etc.

The battery 108 may be configured to provide current to power the electronics 116, which may be used to control operation of the impactor 100. The battery 108 may be coupled to the electronics 116 using one or more wires. Alternatively, or in addition, the battery 108 may wirelessly (e.g., using near-field) supply to the electronics 116. The electronics 116 may include a central processing unit (CPU) that may be arranged and configured to generate and/or execute one or more instructions associated with operation of the impactor 100. The electronics 116 may also include a memory for storing various data, information, etc., such as, for example, instructions for the CPU, a permanent storage for storing larger amounts of information, a communications interface for communicating with one or more components of the impactor 100 or with one or more external devices (e.g., a USB type port, a network connector, etc.), and/or any other components. In some example, alternate implementations, the impactor 100 and/or the electronics 116 may include one or more microcontrollers that may be arranged and configured to control and/or coordinate one or more components of the impactor 100.

In some implementations, as stated above, the impactor 100 may include one or more user interfaces (not shown in FIG. 1) that may be used for inputting various operational parameters and/or changes thereof. For example, the parameters may include, but are not limited to, a speed of an impact, a energy of the impact, a frequency of the impact, a direction of the impact (e.g., whether to drive in an implant or remove an implant), a depth of the implant (e.g., how far into a bone the implant is to be driven), a stability of the implant (e.g., as may be measured by hoop stress), and/or any other parameters. These parameters may relate to a single impact and/or to multiple impacts. In some implementations, the user may input a single parameter, using which, the electronics 116 may be configured to determine any other operations parameters (e.g., input of an energy parameter may lead the electronics determine that positioning of an implant is intended and should be performed at a certain speed).

In some implementations, the user interface may be used for not only inputting operational parameters of the impactor 100, but also for observing its operation and viewing how changes in one or more operational parameters may affect operation of the impactor 100. For example, a change in frequency of the impact may trigger changes (e.g., automatic changes and/or a request for manual changes) in energy of the impact and/or a speed of the impact. The electronics 116 may determine such changes and display changed parameters, along with an any effects on the operation of the impactor 100 and/or implantation/removal process, on the user interface. For example, the effects may include an indication of a change in the remaining battery level, and/or a determined impact stability, a total impact energy that has been transferred to the implant, etc.

In some example, non-limiting implementations, the strike mechanism 118 may be used to actuate positioning and/or removal of an implant by the impactor 100 after receiving one or more instructions from the electronics 116. For example, upon actuating the trigger 106 (e.g., after entry of appropriate operational parameters), the electronics 116 may be configured to generate one or more operational instructions in accordance with one or more operational parameters and provide such instructions to the strike mechanism 118. Upon receiving instructions from the electronics 116, the strike mechanism 118 may be configured to impart energy to the distal connector 110 (positioned within the housing 114), which, in turn, may transfer that energy to an object (e.g., an implant, a tool, etc.) that may be coupled to the distal connector 110 and/or directly to the implant (either for positioning or removal).

In some implementations, the distal connector 110 may include one or more attachment structures and/or mechanism that may be arranged and configured to couple to an object (e.g., drills, cutting tools, effectors, broaches, implants, etc.). For coupling to the distal connector 110, the object (not shown in FIG. 1) may include a click-fit, snap-fit, etc. attachment structure into which the distal connector 110 may mate with. Alternatively, or in addition, the distal connector may include one or more of the following attachment structures and/or mechanisms: a gripping structure (e.g., a clamp), a snap-fit structure, an interference-fit structure, a magnetic attachment mechanism, a releasable attachment mechanism, an interchangeable attachment structure/mechanism, and/or any other type of structures/mechanisms. In some implementations, the distal connector 110 may include a single attachment structure/mechanism and/or multiple attachment structures/mechanism, such as, for example, a snap-fit structure that may be used when implanting an object and a gripping structure that may be used for removing an object. The distal connector 110 may be arranged and configured to be used with a plurality of attachment structures/mechanism may also be beneficial since manufacturers of different implants may require different attachment structure.

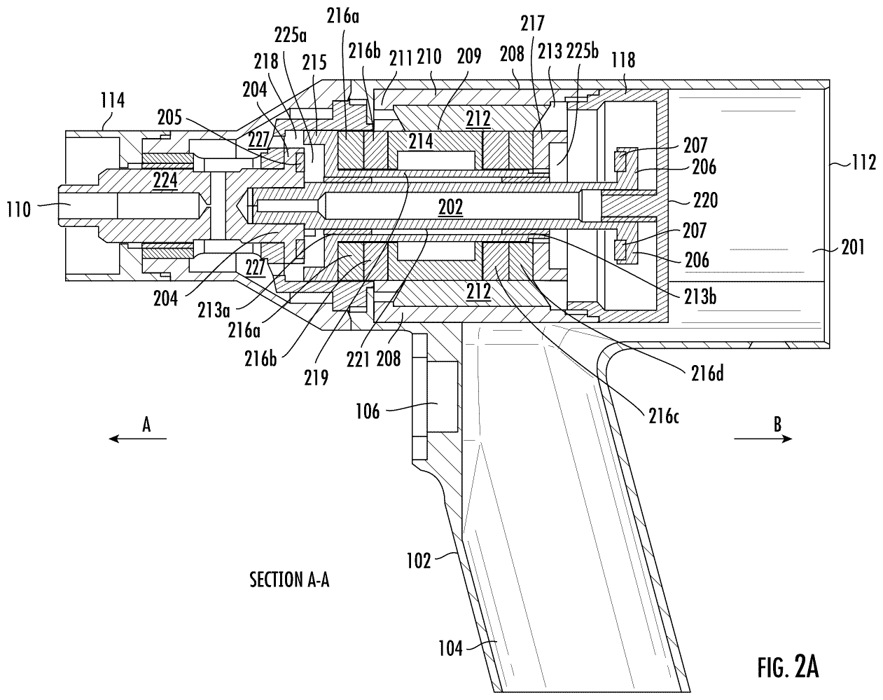

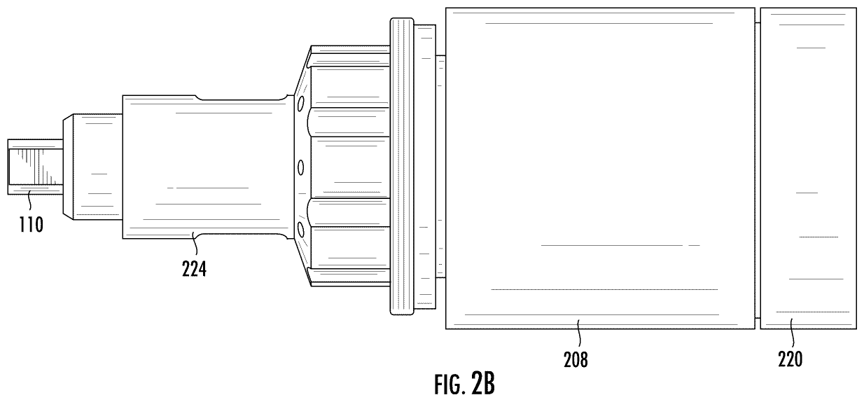

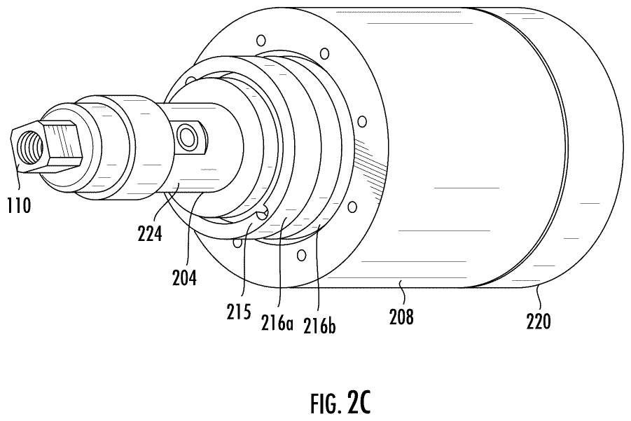

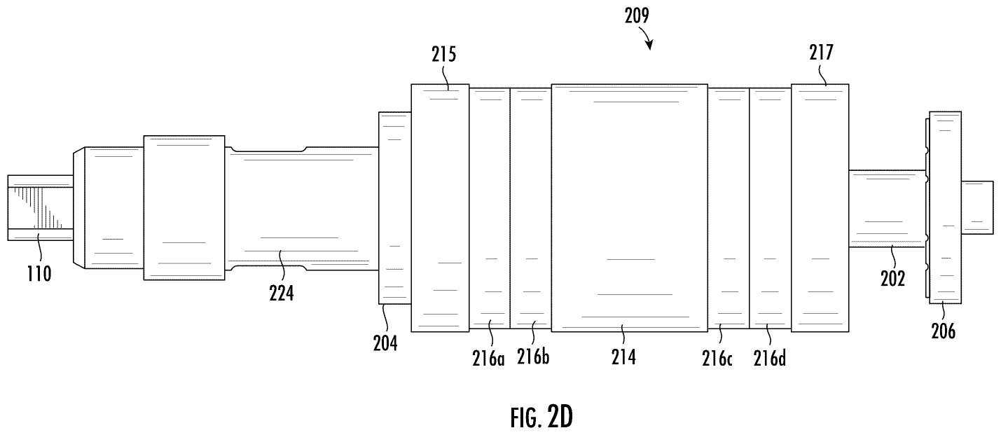

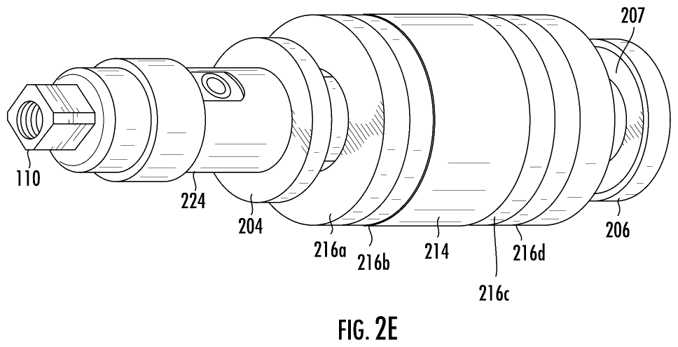

FIGS. 2a-e illustrate further details of the impactor 100 shown in FIG. 1. In particular, FIG. 2a is a cross-sectional view of the impactor 100 shown in FIG. 1, according to some implementations of the current subject matter. The cross-sectional view is taken along sectioning plane A-A shown in FIG. 1 and illustrates the details of the strike mechanism 118. FIGS. 2b-2e are perspective views of the strike mechanism 118.

As stated above, the strike mechanism 118 may be used to interact with the object coupled to the distal connector 110 for the purposes of imparting energy to the object. The energy may, for example, be used to insert an implant into a bone of a patient, and/or to remove an implant from the bone (e.g., to replace it, to correct improper installation, etc.). As can be understood, the energy imparted by the strike mechanism 118 may be used for any other purposes. The strike mechanism 118 may be configured to impart energy as a result of receiving current signals from electronics 116 (not shown in FIG. 2). The current signals may be used to generate an electromagnetic field triggering movement of at least one component of the striker mechanism 118, thereby causing the distal connector 110 to translate.

As shown in FIG. 2a, the strike mechanism 118 may be disposed within an interior portion 201 of the actuator mechanism housing 112. The interior portion 201 may be at least partially hollow so that it may accommodate positioning of the strike mechanism 118. The strike mechanism 118 may include a striker body 202 coupled to the distal connector 110 using an interior connector 224, a distal strike plate 204 coupled to a noise suppression lining 205, a proximal strike plate 206 coupled to a noise suppression lining 207, a stationary electromagnetic housing 208, a moving magnet actuator component 209 and a stop housing 220.

The stationary electromagnetic housing 208 may be configured to remain stationary during operation of the impactor 100 while the moving magnet actuator component 209 moves within an interior of the stationary electromagnetic housing 208 as a result of application of current. The stationary electromagnetic housing 208 may house a coil 212 that may be configured to receive electrical current from a battery (e.g., the power source 108, as shown in FIG. 1) during operation of the impactor. In some example implementations, the housing 208 may include a first portion 210 disposed circumferentially (radially) and adjacent to the coil 212. The housing 208 may also circumferentially surround the coil 212. The housing 208 may also include a second portion 211 and a third portion 213 that may be disposed axially and adjacent to the coil 212. The second portion 211 may be disposed on a distal side of the coil 212 and the third portion may be disposed on a proximal side of the coil 212. As can be understood, while the housing 208 may be composed of multiple portions (e.g., one or more of portions 211, 210, and 213), it may also be a single unitary housing (e.g., portions 211, 210, 213 may be manufactured as a single housing) that may encompass the coil 212, as shown in FIG. 2a.

The components of the housing 208 may also form a hollow channel 218 that may accommodate positioning and translational movement of the moving magnet actuator 209. The moving magnet actuator 209 may translate within the hollow channel 218 between strikes of the distal strike plate 204 and the proximal strike plate 206. In some examples, one or more bearings may be positioned between the stationary component housing 208 and the moving magnet actuator component 209 to provide for smoother translation of the moving magnet actuator component 209 within the hollow channel 218 during operation of the impactor.

The moving magnet actuator component 209 may include one or more magnets (e.g., permanent magnets) 216 (a, b, c, d), a proximal radial pole component 215, an axial pole component 219, and a distal radial pole component 217. The pole components 215, 217, 219 may be configured to have circular shapes and may secure the magnets 216. The pole component 215 may be positioned at a distal end of the moving magnet actuator component 209 and may be shaped to fit over distal strike plate 204 when striking the strike plate 204. The pole component 219 may be positioned at a proximal end of the moving magnet actuator component 209 and may be shaped to fit over the proximal strike plate 206. The pole component 219 may be positioned between the pole component 215 and 217 forming a tray-like structure for positioning the magnets 216. Alternatively, or in addition, the component 219 may be a hollow shaft that may be configured to secure one or more portions of the moving magnet actuator component 209. The magnets may be separated by a pole component 214. As can be understood, the pole components 215, 217, 219 may be configured as a unitary component or as separate components. To provide for magnetic field permeability, the pole components 214, 215, 217, 219 may be manufactured from any magnetically permeable materials, such as, for example, but not limited to, steel, iron, metal, sintered composite materials, and/or any other desired materials.

The pole components 215, 217, 219 may further be configured to form a hollow interior channel 221 that may accommodate positioning of the striker body 202. One or more bearings 213 (a, b) may be positioned between the striker body 202 and outer surfaces of the pole components 215, 217, 219. The bearings 213 may be configured to allow the striker body 202 to be centrally positioned within the hollow interior as well as translate inside the hollow interior channel 221 while maintaining such central positioning. The bearing 213a may be positioned at a distal end of the moving magnet actuator component 209 and proximate to the pole component 215. The bearing 213b may be positioned at a proximal end of the moving magnet actuator component 209 and proximate to the pole component 217. The hollow interior channel 221 may be sized and configured (along with the hollow channel 218) to allow uninterrupted translation movement of the moving magnet actuator component 209 and the striker body 202 relative to one another upon application of current to the coil 212 (in turn, creating an electromagnetic field).

Such translation movement may cause the moving magnet actuator component 209, and in particular, its distal pole component 215, to strike the distal strike plate 204 and/or, in particular, its proximal pole component 217, the proximal strike plate 206. Striking of the plates 204 and/or 206 may cause the striker body 202 and hence, the distal connector 110, to translate. For example, striking of the distal plate 204 by the distal pole component 215 may cause the striker body 202 and the distal connector 110 to translate forward or toward the location of the distal connector 110 (e.g., to insert an implant into a bone). The forward movement direction is illustrated by arrow A in FIG. 2a. Oppositely, striking of the proximal strike plate 206 by the proximal pole component 217 may cause the striker body 202 and the distal connector to translate in reverse or away from the location of the distal connector 110 (e.g., to remove an implant from the bone). The reverse movement direction is illustrated by arrow B in FIG. 2a. As can be understood, “forward” and “reverse” designations are non-limiting and are provided herein for illustrative purposes only.

In some implementations, the strike plates 204 and 206 may be fitted with noise suppression linings 205, 207, respectively. The noise suppression linings 205, 207 may be positioned on the strike plates surfaces that face the respective pole components 215, 217. The linings 205, 207 may be configured to reduce an amount of noise generated by the pole components 215, 217 striking respective strike plates 204, 206. The linings 205, 207 may be manufactured from any desired materials, such as, for example, but not limited to, polymer materials, composite materials, polyethylene, polyurethane, PEEK, PEI, PPS, and/or any other desired material.

In some implementations, to prevent the striker body 202 from moving further into the interior 201 of the housing 202, the strike mechanism 118 may be configured to include the stop housing 220 (as shown in FIGS. 2a-2c). The stop housing 220 may be configured to accommodate positioning of the proximal strike plate 206. During operation of the impactor, and in particular, upon the assembly 222 striking the proximate strike plate 206, the striker body 202 may be configured to move in reverse (e.g., in direction B). As the striker body 202 moves in reverse, it may be configured to be pushed against the stop housing 220, which may prevent further movement of the striker body 202 into the interior 201.

In the moving magnet actuator component 209, the pole component 214 may be configured to be positioned between pairs of magnets 216, as shown in FIGS. 2d-2e. For example, magnets 216a and 216b may be positioned on one or distal side of the pole component 214 with magnet 216b being positioned adjacent to the pole component 214, and magnets 216c and 216d may be positioned on an opposite or proximal side of the pole component 214 with magnet 216c being positioned adjacent to the pole component 214. In some example implementations, the magnets 216 may be permanent magnets, but as can be understood, the magnets 216 may be any other types of magnets. The magnets 216 in each pair of magnets (i.e., 216a-b and 216c-d) may have opposite polarities. For instance, the magnets 216a and 216b may have a north polarity while the magnets 216c and 216d may have a south polarity. Alternatively, the magnets 216a-b may have a south polarity and the magnets 216c-d may have a north polarity. Any other arrangement of magnets' polarities may be possible. For example, the magnets that are positioned adjacent to the pole components 214 (e.g., magnets 216b and 216c) may have the same or different polarity.

Each magnet 216 may have a circular shape (as shown in FIGS. 2d-2e) with an open interior portion (e.g., which may be akin to magnets 216 having a donut shape). Likewise, the pole component 214 may have a similar shape as the magnets. The outer diameters of the magnets 216 and the pole component 214 may be the same and/or different. Similarly, the inner diameters of open interior portions of the magnets 216 and the pole component 214 may be the same and/or different. In some example, implementations, by having the magnets 216 and the pole component 214 have same outer diameters and same inner diameters of their respective interior portions may allow for a smoother translation movements of the moving magnet actuator component 209 channel 218.

The magnet polarities' arrangement may be configured to generate a high magnetic potential in the one or more pole components 214, 215, 217, 219. Once current is supplied to the coil 212, the coil 212 becomes energized. The energized coil 212 may interact with the magnetic potential forcing the moving magnet actuator component 209 to translate within the channel 218. The direction of translation of the moving magnet actuator component 209 may depend on the direction of the current supplied to the coil 212. By way of a non-limiting example, supplying the current in a counterclockwise direction in the coil 212 may cause a forward translational movement of the moving magnet actuator component 209 causing the striker body 202 and connector 110 to translate forward (i.e., in direction A), and supply of the current in a clockwise direction in the coil 212 may cause a reverse translational movement of the moving magnet actuator component 209 (i.e., in direction B). As can be understood, the current subject matter is not limited to the above examples, and the current may be supplied to the coil 212 in any desired direction so that the moving magnet actuator component 209 may be arranged and configured to move in a particular desired direction.

The coil 212 may be encapsulated into the housing 208 (as shown in FIGS. 2b-2c). The housing 208 may have a circular shape with an open interior (e.g., making the housing 208, with the coil 212, to have a donut shape). As can be understood, the housing 208 may have any desired shape. The housing 208 may, for example, be composed of sintered materials, sintered composite materials, sintered metal composite materials, and/or any other type of materials. Use of sintered materials may allow formation of complex geometrical shapes (e.g., housing 208) through the process of forming solid masses of materials through heat and pressure without melting the materials to the point of liquefication. In addition to formation of complex geometrical shapes, sintering also provides for higher purity and lower weight of a final product, higher manufacturing precision and repeatability, as well as an ability to manufacture high melting point metals.

The coil 212 may also be arranged and configured to be positioned proximate to the moving magnet actuator component 209 and, as a result of being positioned within the housing 208, may further be configured to encompass and/or surround the moving magnet actuator component 209. Having the coil 212 positioned proximate to the moving magnet actuator component 209 may allow for generation of an increased electromagnetic field, which, in turn, may allow for a more forceful translational movement of the moving magnet actuator component 209 (either forward or in reverse) as well as the striker body 202 and distal connector 110.

Once the current is supplied to the coil 212, the coil 212 may be configured to produce electromagnetic flux 302. The flux 302 may include an axial flux portion 304 and a radial flux portion 306, as shown in FIG. 3 (illustrating a cross-sectional view of a portion of the impactor 100 shown in FIG. 1).

The axial flux portion 304 may be configured to extend longitudinally through the housing 208 and along the length of the coil 212, as shown by arrow C in FIG. 3. The radial flux portion 306 may be configured to extend transversely through the housing 208 and along the distal end 308 and proximal end 310 of the coil 212. The direction of the radial flux is shown by arrow D.

As discussed above, eddy currents can occur in housing 208 any time there is a change in magnetic flux. For example, an in-rush of current through the coil 212 can create a large flux differential. Such flux differential can create eddy currents. Eddy currents produce counter magnetomotive force(s) slowing the current in-rush and hence, delaying development of the magnetic field that may be desired for the translation movement of the moving magnet actuator component 209. To reduce the occurrence of eddy currents in the electromagnetic housing 208, the housing 208 may be composed of sintered composite materials, e.g., sintered metal composite materials. Through use of the sintered composite materials, the housing may be configured to include flux carrying elements, thereby reducing occurrence of eddy currents. Reduced eddy currents in turn reduce production of counter magnetomotive force(s), thereby allowing more power to be applied to the moving magnet actuator component 209 and hence, the striker component 202 and the distal connector 110. Moreover, use of sintered composite materials for the housing 208 may alleviate production of heat that is typically associated with eddy currents.

Translational movement of the moving magnet actuator component 209 may cause formation of compressed air within various open spaces (e.g., spaces 225 (a, b). In some cases, such compressed air may provide resistance to the movement of the moving magnet actuator component 209 and thereby, reduce an amount of energy that may be imparted by the striker body 202. Alternatively, or in addition, the compressed air, as a result of its compressed nature, may accelerate movement of the moving magnet actuator component 209, and thereby, increase an amount of energy that may be imparted by the striker body 202, which may, in turn, produce unintended results. The compressed air may be vented using one or more pressure relieve valves (e.g., which may be controllable through use of various pressure sensors) and/or into one or more interior spaces 227.

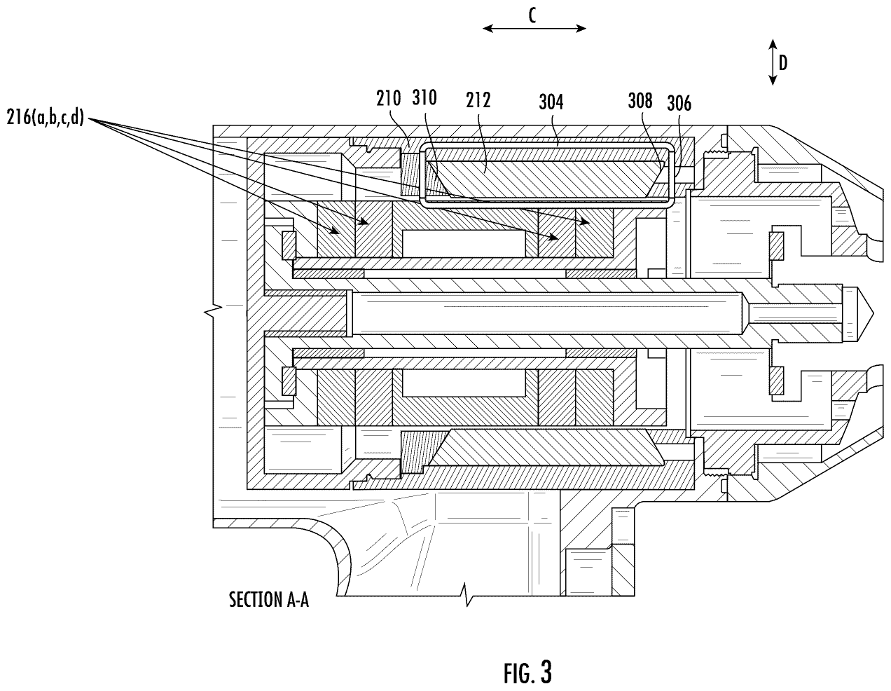

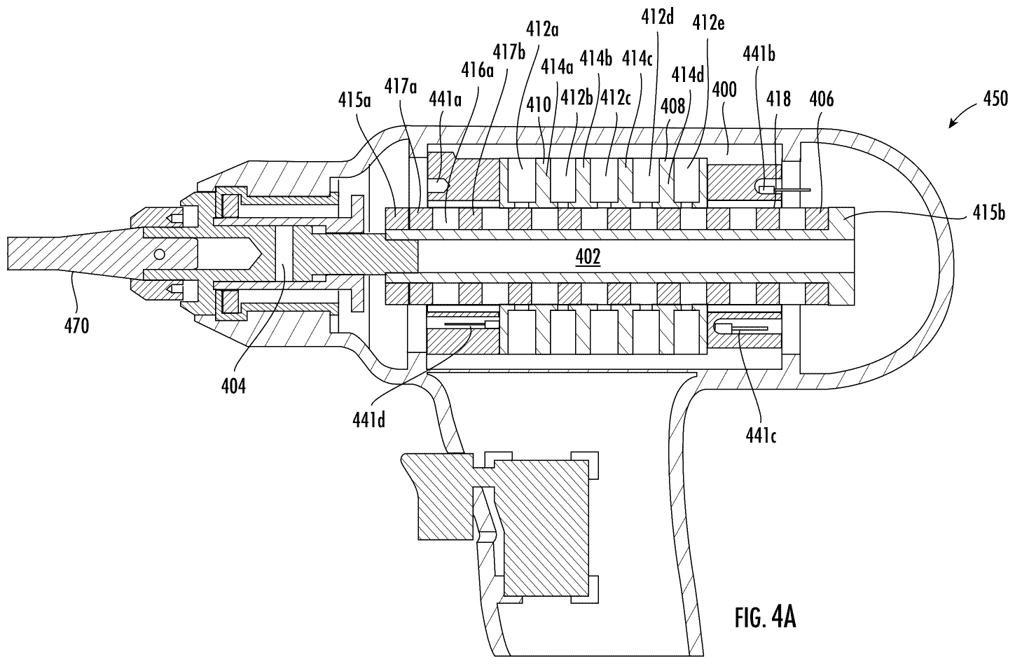

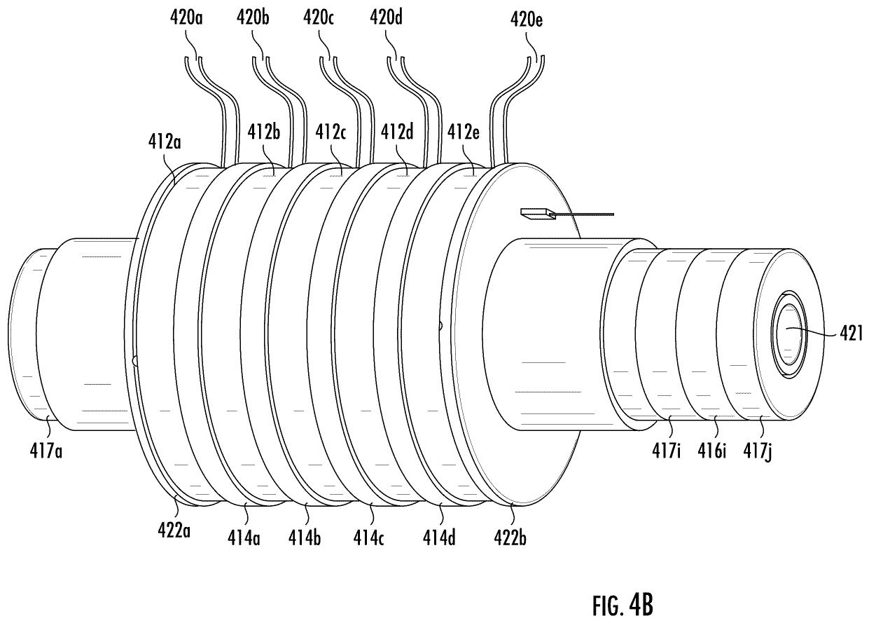

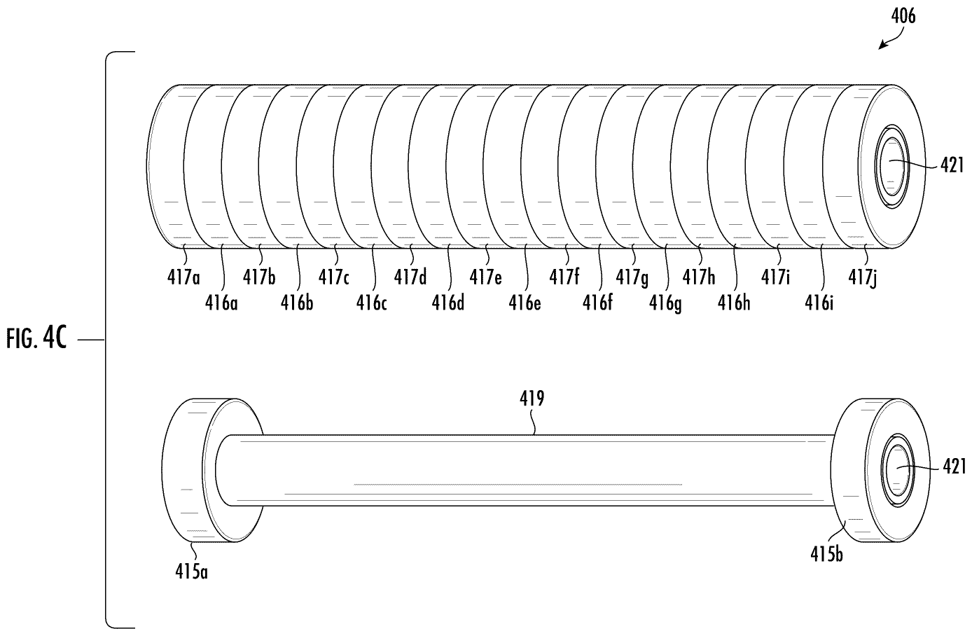

FIGS. 4a-c illustrate cross-sectional views of another example implementation of the impactor 450 according to some implementations of the current subject matter. The impactor 450 may include similar components to the impactor 100 shown in FIGS. 1-3. As shown in FIGS. 4a-b, the impactor 450 may include a linear-motor based strike mechanism 400. FIG. 4a is a cross-sectional view of the impactor with the mechanism 400. FIG. 4b is a perspective view of the mechanism 400. FIG. 4c is a perspective view of the moving magnetic component of the mechanism 400.

Similar to the strike mechanism 118, the strike mechanism 400 may be used to interact with an object that may be coupled to the distal connector 470 for applying or imparting energy on the object, such as, for example, for insertion and/or removal of an implant. To impart energy onto the object, the strike mechanism 400 may be configured to receive current signals from a battery and/or electronic module(s) (not shown in FIGS. 4a-c). The strike mechanism 400 may be similarly arranged within an interior portion of the impactor 450.

The strike mechanism 400 may include a striker body 402 (which may be similar to the striker body 202 shown in FIG. 2a) coupled to the distal connector 470, a strike plate 404 (similar to the distal strike plate 204), a moving magnetic component or armature 406 having one or more magnets 416 (a, b, . . . , i) positioned around the striker body 402, a stationary electromagnetic housing 408, and a coil assembly 410 having one or more coils 412 (a, b, c, d, e) separated by one or more stator components 414 (a, b, c, d).

The stationary electromagnetic housing 408 may be configured to house the coil assembly 410. The housing 408 and the coil assembly 410 may remain stationary during operation of the impactor 450 while the moving magnet actuator component 406 may linearly translate within an interior of the coil assembly 410 as a result of application of current to the coil(s) 412. During operation, one or more coil(s) 412 in the coil assembly 410 may receive electrical current from a battery (e.g., similar to the power source 108, as shown in FIG. 1). In some example implementations, the current may be supplied to each coil 412 independently via one or more respective connectors 420 (a, b, c, d, e) (e.g., connector 420a may be coupled to coil 412a, connector 420b may be coupled to the coil 412b, etc.) that may be coupled to the battery (e.g., via impactor electronic components). The connectors 420 may be wires and/or any other type of connectors. Further, to vary the energy of the impact by the striker 402, the impactor 450 may be configured to selectively supply current to one or more coil, e.g., one or more coils may receive current, while another one or more do not. As shown in FIGS. 4a-b, the coil assembly 410 may include five coils 412, however, as can be understood, the coil assembly 410 may include any number of coils. The number of coils 412 and/or the number to which the current may be supplied during operation may be configured to be proportional to the impact energy delivered by the striker body 402, which, in turn, allows variation of impact energy delivered by the impactor 450 to the object.

As shown in FIG. 4a, the housing 408 may be disposed adjacent to and may circumferentially surround the coil assembly 410. The housing 408 may be composed of multiple portions and/or be single unitary housing that may enclose the coil assembly 410, as shown in FIG. 4a. The housing 408 may also form a hollow channel 418 that may accommodate positioning and translational movement of the moving magnet actuator component 406 along with the striker body 402. The moving magnet actuator component 406 may translate within the hollow channel 418 between strikes. Similar to the impactor 100, the impactor 450 may include one or more bearings and/or bushings (not shown in FIGS. 4a-c) positioned between the stationary component housing 408 and the moving magnet actuator component 406 to provide for smoother translation of the moving magnet actuator component 406 within the hollow channel 418 during operation of the impactor 450.

The housing 408 may have a circular shape with an open interior (e.g., making the housing 408, with the coil assembly 410, to have a donut shape). As can be understood, the housing 408 may have any desired shape. The housing 408 may, for example, be composed of sintered materials, sintered composite materials, sintered metal composite materials, and/or any other type of materials.

As discussed herein, the coils 412 may be separated by the stator components 414. For example, the coils 412a and 412b may be separated by the stator component 414a; the coils 412b and 412c may be separated by the stator component 414b; etc. The coils 412 and stator components 414 may be configured to circumferentially surround the moving magnet actuator component 406, thereby allowing the moving magnet actuator component 406 along with the striker body 402 to translate within the hollow channel 418. The coil assembly 410 may also be enclosed using end portions 422a and 422b. The end portion 422a may be positioned proximate to the coil 412a and the end portion 422b may be positioned proximate to the coil 412e. The end portions 422 may be configured to secure the coils 412 and the stator components 414.

Further, the stator components 414 may have a predetermined width, which allows the coils 412 to be separated a predetermined distance apart. Such separation of coils 412 may be configured to control generation and application of the impact energy by the impactor 450 during operation.

As shown in a top portion of FIG. 4c, the moving magnet actuator component 406 may include one or more magnets (e.g., permanent magnets, magnetized metallic components, etc.) 416 (a, b, . . . , i) separated by one or more pole components 417 (a, b, . . . , j), and enclosed by end portions 415a and 415b. The end portion 415a may be positioned adjacent to the pole component 417a and the end portion 415b may be positioned adjacent to the pole component 417j. The pole components 417 and the magnets 416 may be configured to form pole pairs, where each pole pair may have opposite polarities with the pole pairs adjacent to it. For example, the pole pair formed by the magnet 416a and pole component 417a (e.g., north polarity) may have a different polarity from the pole pair formed by the magnet 416b and the pole component 417b (e.g., south polarity).

In some example implementations, the magnets 416 may be permanent magnets, but as can be understood, the magnets 416 may be any other types of magnets. Further, in some cases, the adjacent magnets 416 (as separated by a pole component 417) may have opposite polarities, while in others, such magnets may have the same polarity. Any other arrangement of magnets' polarities may be possible. For example, the magnets that are positioned adjacent to a pole components 417 may have the same or different polarity.

The magnets 416 and the pole components 417 may be configured to have circular donut-like shapes, where the pole components 417 along with end portions 415 may secure magnets 416 about an internal tube 419 (shown in the bottom portion of FIG. 4c). The internal tube 419 may include a hollow interior 421 that may accommodate positioning of the striker body 402 (not shown in FIG. 4c). The end portions 415 and the internal tube 419 may form a tray-like structure for positioning the magnets 416 and the pole components 417. The outer diameters of the magnets 416 and the pole component 417 may be the same and/or different. Similarly, the inner diameters of open interior portions of the magnets 416 and the pole component 417 may be the same and/or different. In some example implementations, by having the magnets 416 and the pole component 417 have same outer diameters and same inner diameters of their respective interior portions may allow for a smoother translation movements of the moving magnet actuator component 406 withing channel 418. To provide for magnetic field permeability, the pole components 417 may be manufactured from any magnetically permeable materials, such as, for example, but not limited to, steel, iron, metal, sintered composite materials, and/or any other desired materials.

Securing of the striker body 402 inside the hollow interior 421 allows the striker body 402 to translate together with the moving magnet actuator component 406 once current is applied to the coils 412. Such translation movement may cause the striker body 402 to strike the strike plate 404 causing application of energy to the distal connector 470, which, in turn, imparts energy onto the object coupled to the distal connector 470. Similar to the discussion of impactor 100, the impactor 450 may be configured to provide forward and/or reverse impacts (e.g., through forward and reverse translations of the component 406, respectively) on objects coupled to the distal connector 470. Such different direction movements may be accomplished by applying current in different directions to the coils 412. As can be understood, “forward” and “reverse” designations are non-limiting and are provided herein for illustrative purposes only.

In some implementations, to prevent the striker body 402 from moving further into the interior of the housing of the impactor 450, the housing of the impactor 450 may include a stop housing (similar to the stop housing of the impactor 100 shown in FIGS. 2a-2c). The structure and/or operation of the stop housing of the impactor 450 may be similar to the structure and/or operation of the stop housing of the impactor 100.

In some implementations, one or more mechanical, electrical, magnetic, and/or electro-magnetic characteristics of one or more components of the strike mechanism 400 may be specifically selected to allow for an optimal delivery of the impact to the object coupled to the distal connector 470. Such characteristics may include, for example, incorporation of a particular number of coils 412 in the strike mechanism 400 and/or selection of specific coils 412 out of available coils in the strike mechanism 400 to which current is supplied during operation. Additional characteristics may include at least one of: one or more distances between one or more coils 412 (which may vary from coil to coil and/or be uniform), one or more distances between one or more magnets 416 as defined by thicknesses of one or more pole components 417 (which may vary from pole component to pole component and/or be uniform), polarities of one or more magnets 416, and/or any other characteristics.

Once characteristics of the strike mechanism 400 are defined and/or selected, the impactor 400 may be operated. To initiate operation, current may be supplied to the coil(s) 412 from impactor's power source (e.g., a battery), thereby energizing the coil(s). The energized coil(s) 412 may interact with the magnetic potential forcing the moving magnet actuator component 406 to translate along with the striker body 402 within the channel 418. The direction of translation of the moving magnet actuator component 406 may depend on the direction of the current supplied to the coil(s) 412. By way of a non-limiting example, supplying the current in a counterclockwise direction in the coil(s) 412 may cause a forward translational movement of the moving magnet actuator component 406 causing the striker body 402 to translate forward. Oppositely, supply of the current in a clockwise direction in the coil(s) 412 may cause a reverse translational movement of the moving magnet actuator component 406. As can be understood, the current subject matter is not limited to the above examples, and the current may be supplied to the coil(s) 412 in any desired direction so that the moving magnet actuator component 406 may be arranged and configured to move in a particular desired direction. In some implementations, the impactor 450 may be configured to include a predetermined total number of coils 412 (one or more of which may have a predetermined thickness/width) and/or a predetermined total number of poles 417 and/or any combination thereof. Further, in view of the impactor 450 having such predetermined total numbers of coils 412 and/or poles 417, to provide and/or sustain a desired translational movement of the moving magnet actuator component 406 and/or the striker body 402 in a particular direction, each coil 412 and/or one or more coils 412 and/or one or more groups of coils 412 may be supplied a predetermined value of current in one or more specific directions and/or combinations of directions (e.g., clockwise, counterclockwise, etc.) and/or remain unpowered.

In some implementations, the impactor 450 may include one or more sensors 441 (a, b, c, d) for detecting position and/or direction of the moving magnet actuator component 406. The sensors 441 may be disposed proximate to the moving magnet actuator component 406. The sensors 441 may be Hall effect sensors, which may detect magnetic field associated with the component 406 and transmit an appropriate signal to electronics of the impactor 450, which may in turn, use the signal to determine position and/or direction of translation of the component 406. As can be understood, any other way of determining position and/or direction of translation of the component 406 is possible.

The following is a discussion of an exemplary, non-limiting orthopedic surgical instrument and/or impactor that may be used in connection with impactor 100 shown in FIG. 1. As can be understood, the current subject matter is not limited to the use implementation of the impactor and/or any specific tools, and may be used in connection with any desired devices, mechanisms, tools, etc.

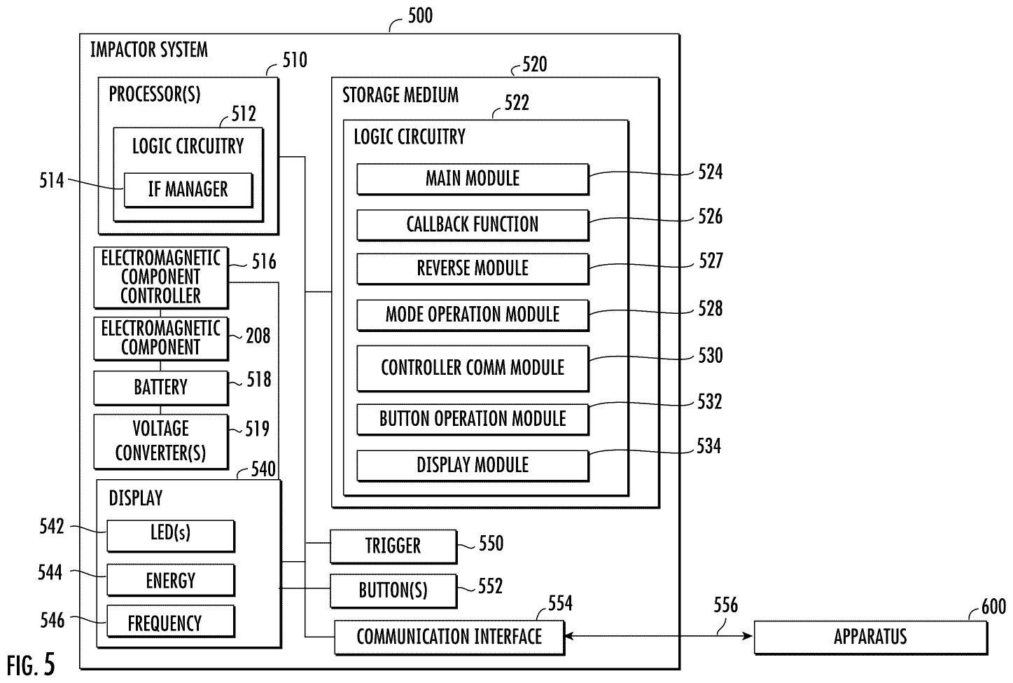

FIG. 5 is a block diagram of an exemplary orthopedic surgical instrument and/or impactor 500, according to some implementations of the current subject matter. The impactor 500 may be similar to the impactor shown and described above in connection with FIGS. 1-3.

The impactor 500 may combine with any suitable example of the systems, devices, and methods disclosed herein. The impactor 500 may include processor(s) 510, a non-transitory storage medium 520, an electromagnetic component controller 516, a battery 518, a voltage converter 519, a display 540, a trigger 550, button(s) 552, and a communication interface 554. The processor(s) 510 may include one or more processors, such as a programmable processor, a micro-controller unit (MCU), and/or the like. The processor(s) 510 may include processing circuitry to implement impactor logic circuitry 512 and 522.

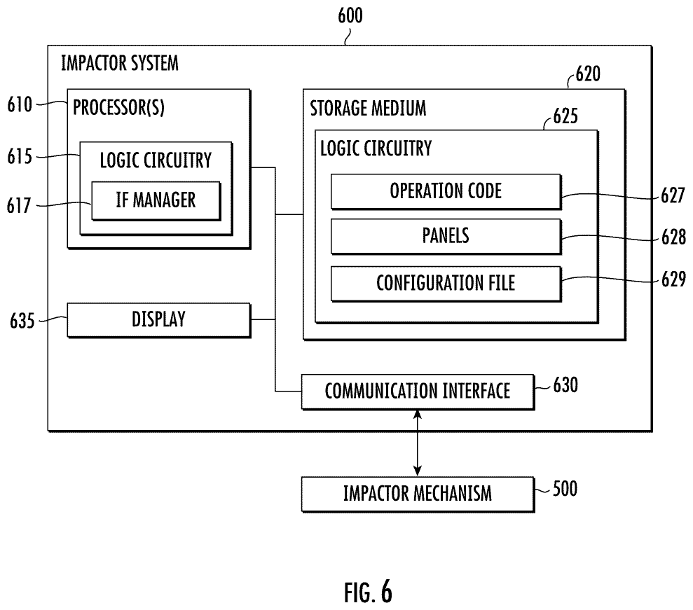

The processor(s) 510 may operatively couple with a non-transitory storage medium 520. The non-transitory storage medium 520 may store logic, code, and/or program instructions executable by the processor(s) 510 for performing one or more operations including the operations of the impactor logic circuitry 522. The non-transitory storage medium 520 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 520 can store logic, code and/or program instructions executable by the processor(s) 510 to perform any suitable implementations of the current subject matter, as described herein. For example, the processor(s) 510 may execute instructions such as instructions of impactor logic circuitry 512 causing the electromagnetic component 208 to operate the impactor at an impact energy and/or frequency selected by a user via button(s) 552 and/or via apparatus 600 (as shown in FIG. 6).

The processor(s) 510 may include code for the impactor 500 in memory within the processor(s) 510 and/or closely connected such as flash memory. The impactor logic circuitry 512 may represent code in or near the processor(s) 512 for execution by the processor(s) 510 and may include a user interface manager 514. The user interface manager 514 may include code executing on the processor(s) 510 to detect and respond to user input as well as to detect the electromagnetic component controller 516 and establish communication with the electromagnetic component controller 516.

The user interface manager 514 may communicate with the electromagnetic component controller 516 to receive status information about the electromagnetic component 208 and to control operation of the electromagnetic component 208. For instance, all button presses of button(s) 552 and edit events may be posted to the user interface manager 514 and processed in real-time. The user interface manager 514 may communicate commands with the electromagnetic component controller 516 to execute in response to the user's actions via button presses, system states, and error conditions. The user interface manager 514 may communicate alerts, warnings, and notifications to a user via the display 540 and or the apparatus 600 (as shown in FIG. 6) via the communications interface 554. Further, the user interface manager 514 may also handle user's response to alerts.

The battery 518 may include any desired power source.

The voltage converter(s) 519 may include a DC-DC voltage converters to adjust the voltage of signals to various voltages required to operate the components of the impactor 500 such as the processor(s) 510, the storage medium 520, and electromagnetic component controller 516, the display 540, the trigger 550, the buttons 552, the communications interface 554, and/or the like.

The storage medium 520 may include a code for execution by the processor(s) 510 to operate the impactor 500. If desired, the processor(s) 510 may copy code from the storage medium 520 to memory closer to the processor(s) 510 to facilitate faster execution of the code. For instance, the user interface manager 514 may include code copied from the impactor logic circuitry 522 to memory closer to the processor(s) 510 for execution.

The impactor logic circuitry 522 may include code for operation of the impactor 500 stored in hardware of the storage medium such as volatile or non-volatile memory in the storage medium 520. The impactor logic circuitry 522 may include a main module 524, a callback module 526, a reverse module 527, a mode operation module 528, a controller communications module 530, a button operation module 532, and a display module 534.

The main module 524 may include setup and loop functions. The setup function may run once at start-up and the loop function may run continuously afterwards. The setup function may attach interrupts that run when button(s) 552 are pressed on the user interface, initializes Timer1 which runs the trigger interrupt service routine (ISR), and initializes an impact delay for the electromagnetic component 208. The loop function allows the electromagnetic component 208 to operate in the user-desired mode when the trigger 550 is enabled and pulled. The loop function also handles showing the user that the trigger state is enabled via LED(s) 542 of the display 540 and/or via the apparatus 600 (shown in FIG. 6).

The callback function 526 may be, e.g., an ISR that runs every millisecond. In some example implementations, the callback function 526 may run periodically with at a time period of more than one millisecond or less than one millisecond.

The reverse module 527 may include functions to prepare to reverse the direction of translation of the electromagnetic component 208, direction change of the electromagnetic component 208, calculate impact delay of the impactor, and setup flutter time delays to set the frequency of impact while in flutter mode. These functions may switch the direction of translation of the electromagnetic component 208, reversing the electromagnetic component 208 to allow for bi-directional operation of the impactor, and may also determine the delay between reversals for controlling a frequency of impacts of the impactor in a flutter mode.

The mode operation module 528 may include the functions of position check, flutter check, and oscillation check functions which are called for normal/full-swing mode, high-frequency/flutter mode, and oscillation mode, respectively. Normal operation checks the position of the electromagnetic component 208 then calls the prepare to reverse function.

The controller communication module 530 may include the functions of enable electromagnetic component controller 516 functions, set current, set current direction, and disable the electromagnetic component controller 516 functions. These functions communicate to the electromagnetic component controller 516 whether or not to operate the electromagnetic component 208.

The button operation module 532 may include functions to handle setting user-desired frequency to operate the electromagnetic component 208 in addition to setting the operation mode and enabling the trigger 550. The functions may include energy plus to increase the energy of impact by the impactor, energy minus to increase the energy of impact by the impactor, frequency plus to increase the frequency of impacts by the impactor, frequency minus to decrease the frequency of impacts by the impactor, select operating mode to switch between available modes of operation (e.g., full-swing mode, flutter mode, or oscillation mode), and set trigger state to enable or disable the trigger 550. In some implementations, these functions may be accessed via the apparatus 600 (shown in FIG. 6) and/or the button(s) 552. In alternate implementations, a touch screen may be included in the display in lieu of or in addition to the button(s) 552.

The display module 534 may include functions handle the logic for displaying the amperage and frequency on the user interface. The functions may include energy display and frequency display.

The display 540 may include LED(s) 540 and numerical, alphanumeric, or graphical displays such as LED displays or liquid crystal displays (LCDs) to present a number representative of the energy 544 and frequency 546 selected for operation of the electromagnetic component 208. The button(s) 552 may include one or more buttons located in the display 540 and, in some implementations, adjacent to the energy 544 and frequency 546 displays to provide a user with an interface to increase and/or decrease the energy and/or frequency of the impact of the impactor on the forward and/or the reverse motion.

The trigger 550 may include a trigger or other button or switch that, when actuated, can cause the impactor 500 to operate if the trigger 550 is enabled. If the trigger 550 is disabled, depressing the trigger 550 may not cause the impactor 500 to operate. In some implementations, the trigger 550 cannot be depressed when the trigger 550 is disabled.

The processor(s) 510 may couple to a communication interface 554 to communicate with an apparatus 600 via a communications medium 556. The communications medium 556 may comprise a wired or wireless interface to communicatively coupled the impactor 500 with the apparatus 600 shown in FIG. 6.

The communication interface 554 may communicate user commands to and/or from the apparatus 600 to the impactor 500 to operate the impactor 500 via the functionality described in conjunction with the impactor 500. In some implementations, the apparatus 500 may operate the electromagnetic component 208 in addition to configuring parameters of operation of the electromagnetic component 208 such as the operating current, the upper frequency bound, the lower frequency bound, the operating frequency, the mode of operation of the electromagnetic component 208, and/or the like. In some implementations, the communication interface 554 may communicate information about the operation of the impactor 500 to the apparatus 600 such as the energy of operation, the frequency of operation, the mode of operation, events or alerts associated with the impactor 500, and log information such as time and date of use, impact detections, encoder counts, and/or the like.

The communication interface 556 (and similarly, communication interface 630 shown in FIG. 6) may include circuitry to transmit and receive communications through a wired and/or wireless media such as an Ethernet interface, a wireless fidelity (Wi-Fi) interface, a cellular data interface, and/or the like. In some implementations, the communication interface 556 (and/or interface 630) may implement logic such as code in a baseband processor to interact with a physical layer device to transmit and receive wireless communications from apparatus 600. For example, the communication interface 556 (and/or interface 630) may implement one or more of local area networks (LAN), wide area networks (WAN), infrared, radio, Wi-Fi, point-to-point (P2P) networks, telecommunication networks, cloud communication, and the like.

FIG. 6 illustrates an exemplary computing apparatus 600, according to some implementations of the current subject matter. The apparatus 600 may be a computing device that may be communicatively coupled with an orthopedic surgical instrument or impactor such as, orthopedic impactor 500 (e.g., as shown in FIG. 5). The apparatus 600 may be a computer in the form of a smart phone, a tablet, a notebook, a desktop computer, a workstation, or a server. The apparatus 600 can combine with any suitable example of the systems, devices, and methods disclosed herein. The apparatus 600 can include processor(s) 610, a non-transitory storage medium 620, communication interface 630, and a display 635. The processor(s) 610 may comprise one or more processors, such as a programmable processor (e.g., a central processing unit (CPU)). The processor(s) 610 may comprise processing circuitry to implement impactor logic circuitry 615 such as the impactor logic circuitry 512 shown in FIG. 5.

The processor(s) 610 may include memory such as flash memory to contain program code for execution by the processor(s) 610. In some implementations, the processor(s) 610 may have random access memory to contain a copy of code from flash memory or read only memory to facilitate faster execution of code. In some implementations, the processor(s) 610 may include cache to contain data for faster calculations or execution. In some implementations, the processor(s) 610 may include an impactor logic circuitry 615, which may include a user interface manager 617. The user interface manager 617 may function as a state machine controlled by keypad inputs, internal events or alarms, boundary conditions, exceptions and supervisory input to the user interface manager 617. The user interface manager 617 may process button presses and may update a main screen on the display 635 reflecting the state of the application.

Upon startup of the user interface manager 617, a handler may be installed to detect the electromagnetic component controller 516 of the impactor 500 and to establish communication with the electromagnetic component controller 516. In some implementations, the button presses of button(s) 552 and edit events may be posted to a panel in the display 635 and may be processed in real-time. Controller commands may be executed upon the user's actions via button presses, system states, and error conditions. Further, the user interface manager 617 may implement alerts, warnings, and notifications and display the alerts, warnings, and notifications via the display 635. The user interface manager 617 may also include code to handle the user's response to alerts, warnings, and notifications.

The processor(s) 610 may operatively couple with a non-transitory storage medium 620. The non-transitory storage medium 620 may store logic, code, and/or program instructions executable by the processor(s) 610 for performing one or more instructions including the impactor logic circuitry 625. The non-transitory storage medium 620 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 620 may store logic, code and/or program instructions executable by the processor(s) 610 to perform any suitable implementation of the methods described herein. For example, the processor(s) 610 may execute instructions such as instructions of impactor logic circuitry 625 causing one or more processors of the processor(s) 610 to communicate user commands to an impactor 500 (as shown in FIG. 5) and/or to communicate events, alerts, operation parameters for the impactor 500, and configurations.

The impactor logic circuitry 625 may include operation code 627, panels 628, and a configuration file 629. The operation code 627 may include functionality to set energy boundaries for operation of the impactor 500, set frequency boundaries for operation of the impactor 500, set an operating energy, set an operating frequency, set an impactor detection profile, set an operating mode (full swing, flutter, or oscillation), and/or the like.

The panels 628 may define graphical user interfaces for display of information and for receiving input parameters or configurations from a user. The configuration file 630 may include user selected parameters such as a controller with which to communicate, boundaries for energy (current), boundaries for frequency of impact, numbers of interrupts expected for push current and for pull current, and/or number of interrupts to receive to establish a frequency of impact.

The processor(s) 610 may couple to a communication interface 630 to transmit the data, code, or commands to and/or receive data, code, or commands from one or more external devices (e.g., a terminal, display device, a smart phone, a tablet, a server, or other remote device). The communication interface 630 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some implementations, the communication interface 630 may implement logic such as code in a baseband processor to interact with a physical layer device to transmit and receive wireless communications from the impactor 500. For example, the communication interface 630 may implement one or more of local area networks (LAN), wide area networks (WAN), infrared, radio, Bluetooth, Wi-Fi, point-to-point (P2P) networks, telecommunication networks, cloud communication, and the like.

The processor(s) 610 may couple to a display 630 to display panels 628 for a user interface and/or other user interface items such as a message or notification via, graphics, video, text, and/or the like. In some implementations, the display 630 may include a display on a terminal, a display device, a smart phone, a tablet, a server, or a remote device.

FIGS. 7-7 illustrate example implementations of a storage medium and computing platform for an orthopedic surgical instrument or impactor in accordance with one or more features of the present disclosure. FIG. 7 illustrates an example of a storage medium 700 to store impactor logic. Storage medium 700 may include an article of manufacture. In some examples, storage medium 700 may include any non-transitory computer readable medium or machine readable medium, such as an optical, magnetic or semiconductor storage. Storage medium 700 may store various types of computer executable instructions 702, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

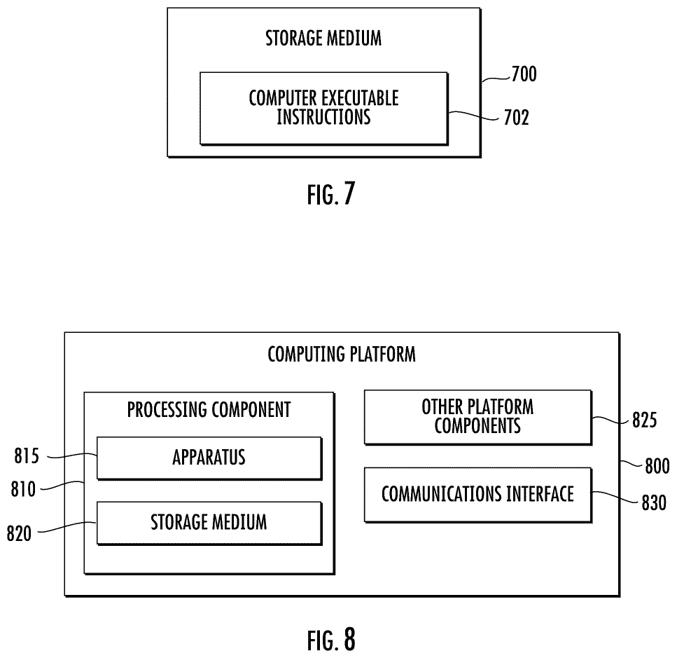

FIG. 8 illustrates an example computing platform 800. In some examples, as shown in FIG. 8, the computing platform 800 may include a processing component 810, other platform components or a communications interface 830. According to some examples, computing platform 800 may be implemented in a computing device such as a server in a system such as a data center or server farm that supports a manager or controller for managing configurable computing resources as mentioned above. Further, the communications interface 830 may include a wake-up radio (WUR) and may be capable of waking up a main radio of the computing platform 800.

According to some examples, processing component 810 may execute processing operations or logic for apparatus 815 described herein such as the impactor logic circuitry 512, 615, and 625 illustrated in FIGS. 5-6. Processing component 810 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 820, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 825 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 830 may include logic and/or features to support a communication interface. For these examples, communications interface 830 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 800 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 800 described herein, may be included or omitted in various implementations of computing platform 800, as suitably desired.

The components and features of computing platform 800 may be implemented using any combination of discrete circuitry, ASICs, logic gates and/or single chip architectures. Further, the features of computing platform 800 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 800 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counterclockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In one example, an impactor may include an electromagnetic component including a stationary electromagnetic housing and a moving magnet actuator component, wherein the stationary electromagnetic housing includes a coil configured to receive an electric current; the moving magnet actuator includes one or more magnets, wherein the one or more magnets are configured to generate a high potential magnetic field that interacts with the electric current applied to the coil and trigger translation movement of the moving magnet actuator component; a striker component configured to be coupled to an object; and one or more strike plates; wherein an electromagnetic field, generated as a result of application of the electric current to the coil, is configured to force the moving magnet actuator component to translate, wherein translation of the moving magnet actuator component is configured to cause the moving magnet actuator component to strike at least one of the strike plates to thereby translate the striker component.

The impactor may include wherein the moving magnet actuator includes two pairs of magnets, wherein each pair of magnets in the two pairs of magnets have an opposite polarity to the other pair of magnets in the two pairs of magnets.

The impactor may include wherein the moving magnet actuator component includes one or more pole components enclosing the magnets.

The impactor may include wherein the pole components are composed of one or more sintered composite materials, the one or more sintered composite materials include sintered metal composite materials.

The impactor may include wherein the stationary electromagnetic housing is composed of one or more sintered composite materials.

The impactor may include wherein directions of translation movement of the moving magnet actuator component and the striker component are directly and/or indirectly dependent on a direction of the current applied to the coil.

The impactor may include wherein a change in a direction of the current applied to the coil is configured to change a direction of translation movement of the moving magnet actuator component and the striker component.

The impactor may include wherein the coil receives electric current causing the electromagnetic component to generate an axial electromagnetic flux and a radial electromagnetic flux.

The impactor may include wherein the sintered composite materials of the housing reduce an occurrence of one or more eddy currents resulting from at least one of the axial electromagnetic flux and/or radial electromagnetic flux.

The impactor may include wherein the sintered composite materials may include sintered metal composite materials.

The impactor may include wherein the one or more magnets are arranged concentrically in relation to the one or more coils.

The impactor may include wherein the one or more magnets are arranged linearly within the stationary electromagnetic housing.

The impactor may include wherein the object includes at least one of: a tool, an implant, and any combination thereof.

The impactor may include wherein the translation movement of the striker component is configured for positioning an implant in a bone and/or remove the implant from the bone.

In one example, a method for using a surgical impactor, wherein the surgical impactor includes an electromagnetic component including a stationary electromagnetic housing and a moving magnet actuator component having one or more magnets, a striker component configured to be coupled to an object, and one or more strike plates, may include applying an electric current to a coil disposed within the stationary electromagnetic housing, wherein the one or more magnets are configured to generate a high potential magnetic field that interacts with the electric current applied to the coil, thereby translating the moving magnet actuator component; triggering, as a result of the applying, translation of striker component, wherein an electromagnetic field, generated as a result of application of the electric current to the coil, is configured to force the moving magnet actuator component to translate, wherein translation of the moving magnet actuator component is configured to cause the moving magnet actuator component to strike at least one of the strike plates to thereby translate the striker component; and performing positioning of the implant in the bone and/or removing of the implant from the bone.

The method may include wherein the moving magnet actuator includes two pairs of magnets, wherein each pair of magnets in the two pairs of magnets have an opposite polarity to the other pair of magnets in the two pairs of magnets.

The method may include wherein the moving magnet actuator component includes one or more pole components enclosing the magnets, wherein the pole components are composed of one or more sintered composite materials, the one or more sintered composite materials include sintered metal composite materials.

The method may include wherein the stationary electromagnetic housing is composed of one or more sintered composite materials.

The method may include wherein directions of translation movement of the moving magnet actuator component and the striker component are directly and/or indirectly dependent on a direction of the current applied to the coil, wherein a change in a direction of the current applied to the coil is configured to change a direction of translation movement of the moving magnet actuator component and the striker component.

The method may include wherein the coil receives electric current causing the electromagnetic component to generate an axial electromagnetic flux and a radial electromagnetic flux, wherein the sintered composite materials of the housing reduce an occurrence of one or more eddy currents resulting from at least one of the axial electromagnetic flux and/or radial electromagnetic flux.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this detailed description by this reference, with each claim standing on its own as a separate implementation of the present disclosure.

What is claimed:

1. An impactor, comprising
an electromagnetic component including a stationary electromagnetic housing and a moving magnet actuator component, wherein
the stationary electromagnetic housing includes a coil configured to receive an electric current;
the moving magnet actuator includes one or more magnets, wherein the one or more magnets are configured to generate a high potential magnetic field that interacts with the electric current applied to the coil and trigger translation movement of the moving magnet actuator component;
a striker component configured to be coupled to an object; and
one or more strike plates;
wherein an electromagnetic field, generated as a result of application of the electric current to the coil, is configured to force the moving magnet actuator component to translate, wherein translation of the moving magnet actuator component is configured to cause the moving magnet actuator component to strike at least one of the strike plates to thereby translate the striker component.

2. The impactor of claim 1, wherein the moving magnet actuator includes one or more pairs of magnets, wherein each pair of magnets in the one or more pairs of magnets have an opposite polarity to at least another pair of magnets in the one or more pairs of magnets.

3. The impactor of claim 1, wherein the moving magnet actuator component includes one or more pole components being positioned adjacent to the magnets.

4. The impactor of claim 3, wherein the pole components are composed of one or more sintered composite materials, the one or more sintered composite materials include sintered metal composite materials.

5. The impactor of claim 1, wherein the stationary electromagnetic housing is composed of one or more sintered composite materials.

6. The impactor of claim 1, wherein directions of translation movement of the moving magnet actuator component and the striker component are directly and/or indirectly dependent on a direction of the current applied to the coil.

7. The impactor of claim 6, wherein a change in a direction of the current applied to the coil is configured to change a direction of translation movement of the moving magnet actuator component and the striker component.

8. The impactor of claim 1, wherein the coil receives electric current causing the electromagnetic component to generate an axial electromagnetic flux and a radial electromagnetic flux.

9. The impactor of claim 8, wherein the sintered composite materials of the housing reduce an occurrence of one or more eddy currents resulting from at least one of the axial electromagnetic flux and/or radial electromagnetic flux.

10. The impactor of claim 9, wherein the sintered composite materials may include sintered metal composite materials.

11. The impactor of claim 1, wherein the one or more magnets are arranged concentrically in relation to the one or more coils.

12. The impactor of claim 1, wherein the one or more magnets are arranged linearly within the stationary electromagnetic housing.

13. The impactor of claim 1, wherein the object includes at least one of: a tool, an implant, and any combination thereof.

14. The impactor of claim 1, wherein the translation movement of the striker component is configured for positioning an implant in a bone and/or remove the implant from the bone.

15. A method for using a surgical impactor, wherein the surgical impactor includes an electromagnetic component including a stationary electromagnetic housing and a moving magnet actuator component having one or more magnets, a striker component configured to be coupled to an object, and one or more strike plates, the method comprising:
applying an electric current to a coil disposed within the stationary electromagnetic housing, wherein the one or more magnets are configured to generate a high potential magnetic field that interacts with the electric current applied to the coil, thereby translating the moving magnet actuator component;
triggering, as a result of the applying, translation of striker component, wherein an electromagnetic field, generated as a result of application of the electric current to the coil, is configured to force the moving magnet actuator component to translate, wherein translation of the moving magnet actuator component is configured to cause the moving magnet actuator component to strike at least one of the strike plates to thereby translate the striker component.

16. The method of claim 15, wherein the moving magnet actuator includes two pairs of magnets, wherein each pair of magnets in the two pairs of magnets have an opposite polarity to the other pair of magnets in the two pairs of magnets.

17. The method of claim 15, wherein the moving magnet actuator component includes one or more pole components enclosing the magnets, wherein the pole components are composed of one or more sintered composite materials, the one or more sintered composite materials include sintered metal composite materials.

18. The method of claim 15, wherein the stationary electromagnetic housing is composed of one or more sintered composite materials.

19. The method of claim 15, wherein directions of translation movement of the moving magnet actuator component and the striker component are directly and/or indirectly dependent on a direction of the current applied to the coil, wherein a change in a direction of the current applied to the coil is configured to change a direction of translation movement of the moving magnet actuator component and the striker component.

20. The method of claim 15, wherein the coil receives electric current causing the electromagnetic component to generate an axial electromagnetic flux and a radial electromagnetic flux, wherein the sintered composite materials of the housing reduce an occurrence of one or more eddy currents resulting from at least one of the axial electromagnetic flux and/or radial electromagnetic flux.
